(19) United States
(12) Patent Application Publication
(10) Pub. No.: US 2017/0071794 A1
(43) Pub. Date: Mar. 16, 2017

(54) Seamless Compression Sleeve

(71) Applicant: SIGVARIS AG, St. Gallen (CH)

(72) Inventors: Xavier Bazoud, Saint Romain Le Puy (FR); Julien Duhem, Saint-Etienne (FR)

(21) Appl. No.: 15/120,393

(22) PCT Filed: Mar. 3, 2015
(86) PCT No.: PCT/EP2015/054410

§ 371 Date: Dec. 2, 2016

(30) Foreign Application Priority Data
Mar. 3, 2014 (FR) 1451673

(51) Int. Cl. A61F 13/10; B29C 65/02; D04B 1/18; A61F 13/00; D04B 1/28

(52) U.S. Cl. CPC A61F 13/104; A61F 13/00987; D04B 1/28; D04B 1/18; B29C 65/02; A61F 2013/00093; A61F 2013/00119; A61F 2013/00238; A61F 2013/0028; B29K 2077/00

(57) ABSTRACT

An orthotic compression brace includes a tubular sleeve namely knit from elastic yarns, covering at least the arm and the hand, up to the insertion of phalanges on the metacarpal bones of the hand, and a protrusion aimed to, at least partially, cover the thumb over its entire circumference. The tubular sleeve and the thumb are knit into one piece and the thumb is knit at least in part as from the thermobonding at a T1 temperature the sleeve eventually containing thermobonding yarns at a T2 temperature strictly greater than T1.

### CROSS-REFERENCE TO RELATED APPLICATIONS

This application is a national stage application under 35 U.S.C. §371 and claims the benefit of priority of international application no. PCT/EP2015/054410, filed Mar. 3, 2015, which claims the benefit of priority under 35 U.S.C. §.119 of French patent application no. 1451673, filed Mar. 3, 2014, the entire contents of each being hereby incorporated herein by reference, in its entirety and for all purposes.

### TECHNICAL AREA

The present disclosure relates to an orthotic compression brace or splint for the treatment of lymphedema in the upper limbs and more particularly the arm. To be more specific, the disclosure relates to a compression sleeve which is entirely seamless so as, namely, to improve patient comfort for those wearing such compression sleeves.

### BACKGROUND

Lymphedema is a swelling in a more or less important part of the body following an accumulation of lymphatic fluid in the connective tissues. Lymphedema appears when the lymphatic vessels do not exist or when the lymphangions of which it is made up become degraded or non-functional. Lymphedema can also occur when the lymphatic vessels are damaged, clogged up or when the lymph nodes have been removed due to some type of damage or trauma caused by an accident, surgery, severe infection or radiotherapy. Besides the fact that lymphedema can lead to severe functional handicap and a marked impairment of quality of life, it can also cause complications such as erysipelas, a bacterial skin infection caused by streptococcus.

The treatment of lymphedema is initially to practice a manual lymphatic drainage procedure called DLM then, secondly, to wear a compression brace such as a compression stocking for the lower limbs or a compression sleeve for the arms.

Document WO00/67600 describes a glove with the end cut off all the fingers, including the thumb. This glove has areas of varying extensibility connected to the rest of the seamless glove. The glove is manufactured in its entire form, for example, from a single blend of nylon and Lycra®.

Document WO2006/01559A1 describes an orthopedic product known as a “spacer”. This article is produced on an assembly line. It combines two outer layers each connected by an intermediary layer. In a particular embodiment, it is in the form of a tube covering the forearm and hand. An opening is formed in the tube wall for the passage of the thumb.

Also known are compression sleeves for the treatment of lymphedema of the arm. They consist of a tubular sleeve knit from elastic yarn covering at least the arm and the hand, and at least up to the insertion of the phalanges on the metacarpal bones of the hand, and an open thumb. A sleeve of this type is for example described in DE202012004652U1.

Lymphedema involving more or less important changes in the volume of the hand and/or fingers and the production of compression sleeves adapted to patient treatment require article precision cutting. So as to achieve this, the current technique consists of obtaining the volume of the thumb by using a knitted compression yoke independently of the main compression section and of using a classical assembly technique by crafting it so as to join together the tubular sleeve covering the arm and the rest of the hand. The most obvious and traditional assembly solution of both these elements consists in the production of a seam at the junction of the thumb and the main part.

Both these parts, the tubular sleeve and the thumb piece, being initially produced independently of each other, may involve varying compression levels. Furthermore, at the seam, product compression is no longer controlled as, in this area, there exists external material with radically different mechanical characteristics from those of the used compression yokes.

Moreover, the area consisting of the seam at the junction of the thumb and the tubular sleeve more than often generates a thickening often causing discomfort due to the superposition of both parts. This discomfort is accentuated since the seam area is in a very mobile area of the hand. The level of discomfort varies from patient to patient; nevertheless, said discomfort emanates from the friction due to the rigidity of the sewing thread used, the excess thickness introduced at the compression level and a certain cutaneous sensitivity linked to the pathology in question.

Finally, wearing a compression sleeve to counteract lymphedema of the hand on occasions turns out to be cumbersome in respect of the carrying out of daily tasks. The patient sometimes has a tendency to free up his hand without completely removing the compression sleeve by stretching forward where the support point is often situated at the very end of the thumb. To successfully remove the compression sleeve, he should then pull it back forcefully, causing very strong tensions insofar as the latter should be higher than the initial compression level so as to be able to free up the compromised limb. These strong mechanical constraints sometimes repeated several times a day, end up weakening the thumb junction/main part sometimes involving tears be it at the level of the seam, this being the most common, or at the level of the fabric close to the seam.

Furthermore, there exists the problem of the finish of the thumb part insofar as the end part of the latter should be open to allow the passage of said thumb. The opening is made by cutting with a sharp element which inevitably leads to a weakening of the mesh with a subsequent risk of unraveling.

### SUMMARY

One goal of the disclosed embodiments is to thus overcome such disadvantages by offering a compression brace for the treatment of lymphedema of the hand by means of a simple and inexpensive design, improving wearability and wear resistance.

Another objective is to develop a brace which has no risk of unraveling at the distal end of the thumb after cutting.

To this end, there is on offer a compression brace comprising a tubular sleeve knit from elastic threads or yarn namely, covering at least the arm and the hand, at least until the insertion of the phalanges on the metacarpal bones of the hand, and a projection aimed at, at least, partially covering the thumb over its entire circumference, the tubular sleeve and the thumb being knit into one piece.

The brace is remarkable in that the thumb is knit in an overall beneficially truncated shape, at least in part as from thermobonding yarn(s) at a T1 temperature, the sleeve eventually containing thermobonding yarn at a T2 temperature strictly greater than T1.

In reality, the thermobonding yarns contained in the protrusion are fusible at a determined T1 temperature in such a way that, post heating the brace to a temperature above the melting temperature of said thermobonding yarns to a T1 temperature, they stick together. Under these conditions, after cooling, the distal end of the thumb can be cut with no untimely and undesired unraveling In the event that the sleeve itself contains thermobonding yarns, these are fusible at a temperature strictly above T1 so that at T1, they retain their original structure and do not stick together.

Again, in reality, the T1 temperature is between 100° C. and 130° C., preferably between 105° C. and 115° C., and, even more preferably, around the 110° C. mark. As will subsequently be seen, these temperatures correspond in real terms to the highest temperatures implemented during the manufacturing process of the orthotic compression brace.

Furthermore, it is well understood that contrary to the compression braces of the prior art comprising a seam connecting the parts of the thumb to the tubular sleeve, the orthotic compression brace has no seam so that patient comfort and wear resistance are improved.

Even more beneficially, the sleeve and protrusion contain the same mixture of textile fibers with the exception of the thermobonding yarn at the T1 temperature solely present in the protrusion. In practice, the textile fibers which make up the sleeve and the projection are selected from the group comprising modal, viscose, elastane, and polyamide. In one advantageous embodiment, the brace is knit from a mixture of modal, elastane, and polyamide. Of course, depending on the areas of the sleeve, the fiber characteristics may vary, such as, for example, their diameter. When the orthotic brace contains polyamide yarns in addition to thermobonding yarn at a T1 temperature, these polyamide yarns are heat-activated at a T2 temperature strictly higher than T1.

According to a first embodiment, the protrusion comprises two non-symmetrical parts knit into one piece, a first part relating to the palm, extending onto the palmar fascia of the thumb when the orthotic brace is worn, and a second part known as dorsal, extending onto the dorsal fascia of the thumb when the orthotic brace is worn, presenting a width of the upper base to the width of the base of the palmar section and a height greater than the height of the palmar section.

Preferably, the ratio between the width of the base of the palmar section of the thumb and the width of the orthotic brace is understood to be between 0.25 and 0.35.

Moreover, the base of the palmar section of the thumb comprises between 80 and 90 meshes, the base of the dorsal portion of the thumb comprises between 82 and 92 meshes, and the distal end before cutting the dorsal part and the palmar section of the thumb comprises between 26 and 36 meshes.

In a second embodiment, the projection includes two non-symmetrical parts knit into one piece.

The thermobonding yarn at a T1 temperature is preferably made up of copolyamide thread.

Finally, so as to allow the passage of the thumb at the time of its introduction, the distal end of the projection is cut away

Another purpose relates to a method of manufacturing a compression orthosis comprising a tubular sleeve knit out of elastic, namely covering at least the arm and the hand, at least up to the insertion of the phalanges on the metacarpal bones of the hand, and a protrusion at least partially covering the thumb over its entire circumference, the tubular sleeve and the thumb being knit into one piece.

The procedure is remarkable in that it comprises at the very least the following steps:
Knitting a tubular sleeve from the thermobonding yarns namely on a circular knitting machine,
Knitting a globally advantageous truncated protrusion at the distal end of said sleeve, said projection being knit from at least a part of the thermobonding yarn(s) at a T1 temperature and being aimed at at least partially covering the thumb (2) over its entire circumference,
Heating the orthotic brace to a temperature higher than the melting point of the thermobonding yarns at a T1 temperature so as to melt and paste said thermobonding yarns,
Cooling the orthotic brace.

The stage of heating the orthotic brace consists of a pre-affixing stage at a T1 temperature. In practice, the T1 temperature is between 100° C. and 130° C., preferably between 105° C. and 115° C., ideally to the order of about 110° C. The pre-affixing is beneficially conducted for a period of between 1 and 2 minutes.

The method beneficially comprises a step of dyeing the orthotic brace between the prefixation stage and the cooling stage. This stage is conducted at a temperature lower than T1, in practice between 80° C. and 98° C., and preferably at about 95° C.

According to another characteristic, the method further contains a cutting stage of the distal end of the thumb after cooling. In reality, cutting is carried out by means of a cutting tool.

Furthermore, the knitting stage of the protrusion forming the thumb consists of knitting into both symmetrical and non-symmetrical parts in one stage.

When the parts are non-symmetrical, the knitting stage of the protrusion forming the thumb consists of knitting into one piece, a first part known as palmar, extending onto the palmar fascia of the thumb when the orthotic brace is worn, and a second part known as dorsal, extending onto the dorsal fascia of the thumb when the orthotic brace is worn, introducing a width of the upper base greater than the width of the base of the palmar part and a height greater than the height of the palmar part.

Preferably, the ratio between the width of the base of the palmar part of the thumb and the width of the orthotic brace should be between 0.25 and 0.35, the base of the palmar part of the thumb should knit on 80-90 needles, and preferably on 85 needles, the base of the dorsal part of the thumb should be knit on 82-92 needles and, preferably, on 87 needles, and the proximal end of the dorsal part and the palmar part of the thumb should be knit on 26-36 needles and, preferably, on 31 needles.

In addition, the thermobonding yarn at the T1 temperature preferably should consist of a copolyamide thread as previously described.

### BRIEF DESCRIPTION OF THE FIGURES

Other advantages and characteristics become apparent from the following description of several alternative embodiments, given by way of non-limiting examples of the compression brace for the treatment of lymphedema of the hand, as from the attached drawings on which:

FIG. 1 is a perspective view of the compression brace view for the treatment of lymphedema of the hand,

FIG. 2 is a top view of the compression brace for the treatment of lymphedema of the hand worn by a patient,

FIG. 3 is a schematic representation of the thumb knitting pattern of the orthotic compression brace for the treatment of lymphedema of the hand,

FIG. 4 is a graphical representation of the breaking force in terms of the circumference of the thumb of several compression orthotic braces for the treatment of lymphedema of the hand,

FIG. 5 is a graphical representation of the breaking force in terms of the circumference of the thumb of several orthotic compression braces of the prior art comprising a sewn-in thumb on the tubular sleeve,

FIG. 6 is a graphical representation of the average breaking force in terms of the circumference of the thumb of the orthotic compression brace and orthotic compression braces of the prior art.

### DETAILED DESCRIPTION

Following the description of the compressive orthotic brace for the treatment of lymphedema of the hand, the same numerical references apply to the same elements. On the other hand, the various views are not necessarily drawn to scale.

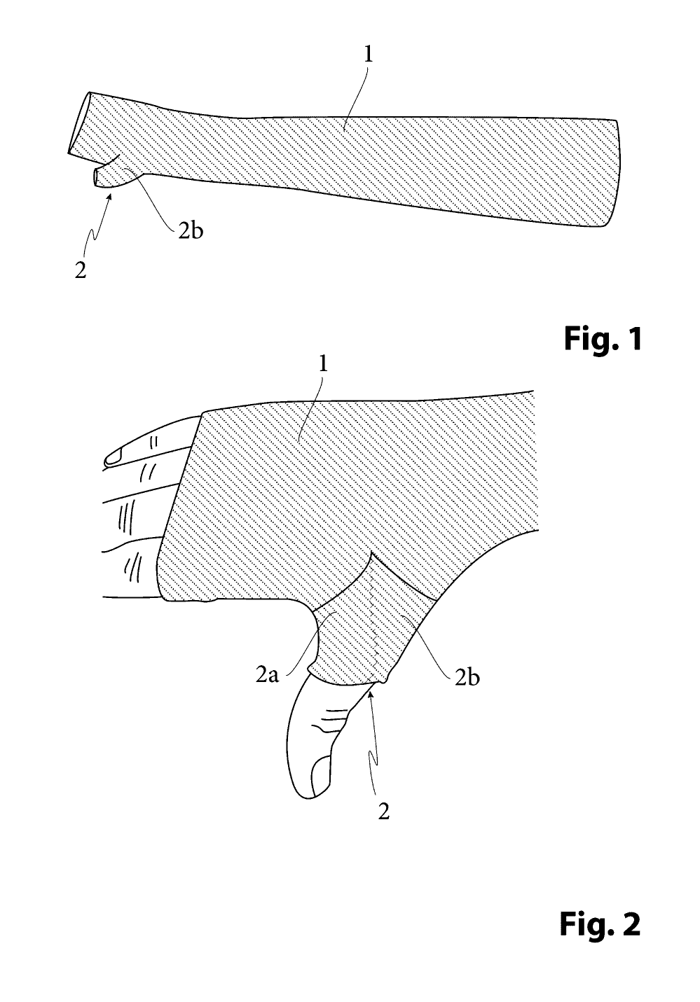

Referring to FIGS. 1 and 2, the compression brace comprises a tubular sleeve (1) knit from elastic threads covering at least the arm and the hand, at least up to insertion of the phalanges on the metacarpal bones of the hand, and a projection aimed at covering at least partially and over all the whole circumference the thumb (2). The tubular sleeve (1) and the thumb (2) are knit into one piece on a circular knitting machine or any other equivalent machine. Said thumb (2) is knit in the form of an overall truncated item as from at least in part of the thermobonding yarns at a predetermined T1 temperature in such a way that, after heating of the orthotic brace at a temperature equal to T1, the distal end of the thumb can be cut without inadvertent unraveling after cooling. Said heat-bonding yarn consists of a copolyamide thread with a melting point less than 130° C. Said thermobonding yarn can, for example, consist of a yarn marketed by EMS-GRILTECH under the reference GRILON® K-110.

It is obvious that the thermobonding yarn may well be made up of any other thermobonding yarn well known in the art without departing from the scope of the disclosed embodiments.

Moreover, those skilled in the art will easily adapt the thermobonding yarn diameter depending on the desired texture of the thumb in particular.

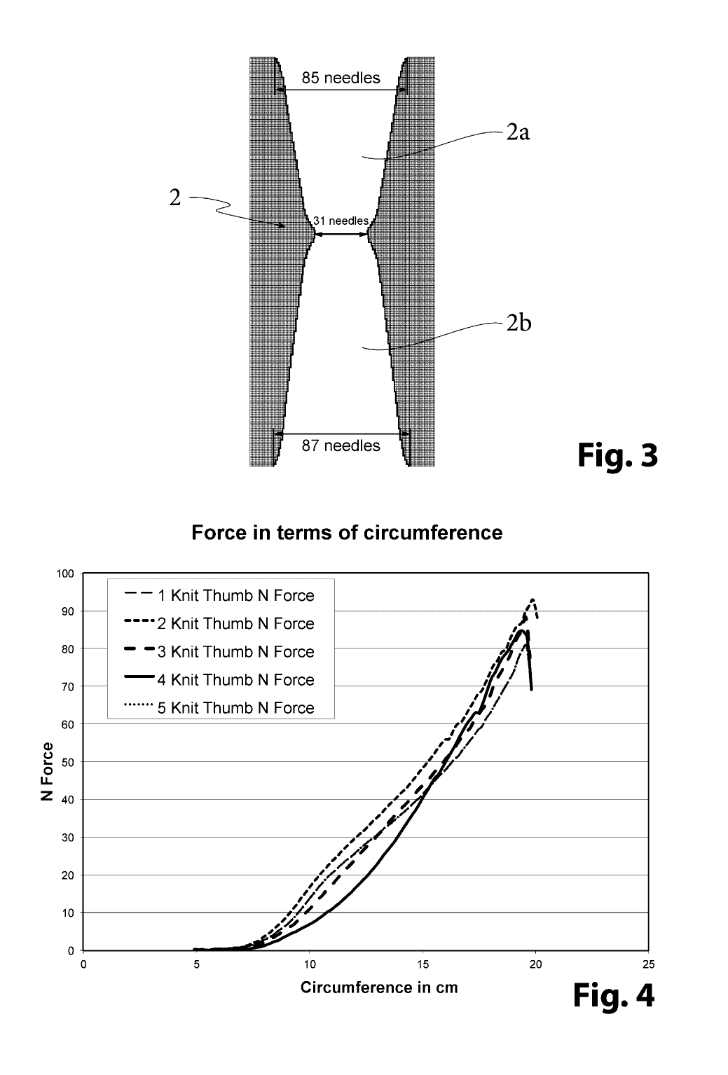

Referring to FIGS. 1 to 3, the thumb (2) includes two non-symmetrical parts (2a, 2b) knit into one piece, a first part known as palmar (2a) extending on the palmar fascia of the thumb when the orthotic brace is worn, and a second part known as dorsal (2b) extending onto the dorsal side of the thumb when the brace is worn. The second dorsal part (2b) has a width of the upper base to the width of the base of the palmar portion (2a) and a height greater than the height of the palmar part (2a). The ratio between the width of the base of the palmar part (2a) of the thumb (2) and the width of the brace is preferably between 0.25 and 0.35. The base of the palmar part (2a) of the thumb (2) comprises between 80 and 90 meshes and the base of the dorsal part (2b) of the thumb (2) comprises between 82 and 92 meshes, with reference to FIG. 3, the tubular sleeve (1) having approximately 290 meshes. Furthermore, the distal end of the dorsal part (2b) and the palmar part (2a) of the thumb comprise between 26 and 36 meshes. In addition, the height of the dorsal part (2b) is greater than the height of the palmar part (2a) of the thumb (2), the dorsal part (2b) preferably has a height of between 140 and 160 rows of mesh and the palmar part (2a) preferably a height of between 110 and 120 mesh rows.

Incidentally, the palmar part (2a) and/or the dorsal part (2b) may include transverse lines, printed or made by means of colored yarns, forming one or several markers for a patient to cut, and ultimately open, the distal end of the thumb (2).

It will be observed that the distal end of the thumb (2) may also be precut without departing from the scope of the disclosed embodiments. In reality, the cutting of the thumb can be performed either directly by the manufacturer of the orthosis, or by the patient, under its doctor's direction. The advantage of the latter is that the patient may accurately adjust the thumb size.

According to the disclosed embodiments, the remainder of the orthosis, namely the sleeve and the remainder of the thumb, is knit from a mixture of modal, elastane, and fusible polyamide at a T2 temperature strictly speaking higher than T1. Consequently, there is no risk of bonding the threads together at the time of heat sealing of the constituent thumb threads that is to say at the pre-affixing stage.

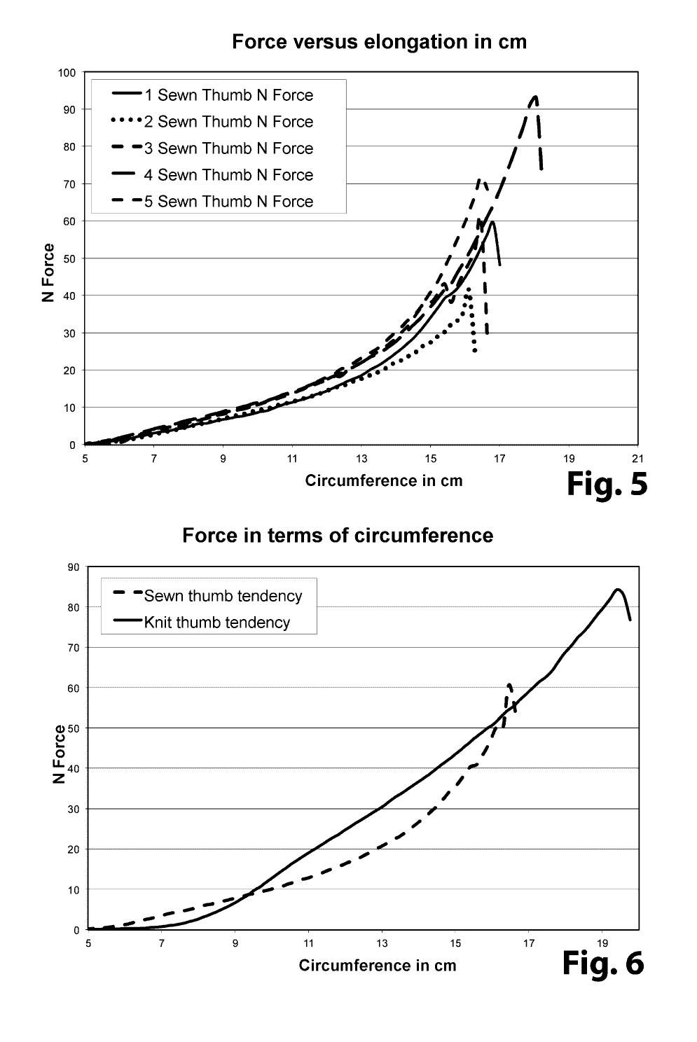

It will be noted that in view of the absence of sewing between the thumb (2) and the tubular sleeve (1), the patient wearing the orthosis will not be particularly hindered by the increased thickness of the seam. Moreover with reference to FIGS. 4-6, it is clearly apparent that the breaking force of the orthotic braces, which have no seam, is greater than the breaking force of the orthotic braces of the prior art which have a seam. Indeed, it can be ascertained, with reference to FIG. 6, that the breaking force of braces is greater by around 16% than the breaking force of the orthotic braces of the prior art. The measurements of the breaking force were carried out using a dynamometer, the breaking force being the force at which is measured a fall of more than 20% in relation to the value of the force previously measured.

The following will describe the manufacturing process of the compression brace. Said method comprises at least the following steps of knitting a tubular sleeve (1) namely from elastic threads, on a circular knitting machine, knitting an overall truncated protrusion at the distal end of said sleeve (1), said protrusion forming the thumb (2) of the brace, as from at least one heat sealing thread at a T1 temperature, and heating the brace to a temperature above melting point of the heat sealing threads at a T1 temperature so as to melt and stick together the aforementioned heat sealing yarns.

Out of preference, the heating stage of the orthotic compression brace to make the heat sealing yarn melt is made up of a pre-affixing stage at a T1 temperature. In this particular embodiment, the thermobonding yarn at a T1 temperature consists of a copolyamide thread whose melting point is around 125° C., marketed by EMS-GRILTECH under the reference GRILON (TMK) K-110; it is, however, obvious that the thermobonding yarn may be included in any heat sealing yarn known to the skilled person in the art and whose melting point is preferably less than or equal to 130° C.

The knitting stage of the protrusion shaping the thumb consists of knitting two non-symmetrical parts into one piece, a first part known as palmar (2a) extending on the palmar fascia of the thumb when the brace is worn and a second part known as dorsal (2b) extending onto the dorsal side of the thumb when the brace is worn, introducing a width of the upper base to the width of the base of the palmar part (2a) and a height greater than the height of the palmar part (2a). The ratio between the width of the base of the palmar part of the thumb and the width of the brace is between 0.25 and 0.35. The base of the palmar part (2a) of the thumb (2) is knit on 80 to 90 needles, and preferably on 85 needles, and the base of the dorsal part (2b) of the thumb (2) is knit on 82-92 needles and, preferably, on 87 needles. The proximal end of the dorsal part (2b) and the palmar part (2a) of the thumb (2) is knit on 26-36 needles and, preferably, on 31 needles.

Finally, it is obvious that the examples that have just been given are only specific illustrations and are, in no case, restrictive as to the scope of the claims.

1. An orthotic compression brace comprising a tubular sleeve namely knit from elastic yarn, covering at least the arm and the hand, at least up to the insertion of the phalanges on the metacarpal bones of the hand, and a protrusion intended to at least partially cover the thumb over its entire circumference, the tubular sleeve and the thumb being knit into one piece, wherein the thumb is at least knit in part from the thermobonding yarn at a T1 temperature.

2. An orthotic compression brace according to claim 1, wherein the T1 temperature is between 100° C. and 130° C.

3. An orthotic compression brace according to claim 1, wherein the sleeve and the projection contain the same mixture of textile fibers with the exception of thermo-bonding yarn at the T1 temperature solely present in the protrusion.

4. An orthotic compression brace according to claim 1, wherein the textile fibers constituting the sleeve and the projection are selected from the group comprising modal, viscose, elastane, and polyamide.

5. An orthotic compression brace according to claim 1, wherein the fusible thread at T1 consists of a copolyamide thread.

6. An orthotic compression brace according to claim 1, wherein the distal end of the protrusion is cut away.

7. A manufacturing process for producing an orthotic compression brace comprising a tubular sleeve namely knit from elastic yarn, covering at least the arm and the hand, at least up to the insertion of the phalanges on the metacarpal bones of the hand, and a protrusion intended to at least partially cover the thumb over its entire circumference, the tubular sleeve and the thumb being knit into one piece, characterized in so far as the thumb at least knit in part from the thermobonding yarn at a T1 temperature, the method comprising the following stages:
Knitting a tubular sleeve namely from elastic thread, on a circular knitting machine;
Knitting a protrusion at the distal end of said sleeve, said projection being knit from, at least in part, thermobonding yarns(s) at a T1 temperature and being destined to at least partially cover the thumb over its entire circumference;
Heating the orthosis to a temperature above melting point of the thermobonding yarns at a T1 temperature so as to melt and stick together the aforementioned thermobonding yarns; and
Cooling the orthotic brace.

8. A manufacturing process according to claim 7, wherein the heating stage consists of a pre-affixing stage at a T1 temperature.

9. A manufacturing process according to claim 7, further comprising a dyeing stage between the pre-affixing stage and the cooling stage.

10. A manufacturing process according to claim 9, wherein the dyeing stage is conducted at a temperature lower than T1.

11. A manufacturing process according to claim 9, wherein the temperature is between 80° C. and 98° C.

12. A manufacturing process according to claim 7, further comprising a cutting stage of the distal end of the thumb.

13. An orthotic compression brace according to claim 1, wherein the sieve eventually contains thermobonding yarn at a T2 temperature strictly higher than T1.

14. A manufacturing process according to claim 7, wherein the sleeve eventually contains thermobonding yarn at a T2 temperature strictly higher than T1.

15. An orthotic compression brace according to claim 1, wherein the T1 temperature is between 105° C. and 115° C.

16. An orthotic compression brace according to claim 1, wherein the T1 temperature is around 110° C.

17. A manufacturing process according to claim 9, wherein the temperature is about 95° C.
